(19) United States
(12) Patent Application Publication
(10) Pub. No.: US 2018/0140723 A1
(43) Pub. Date: May 24, 2018

(54) OPTIMISED MULTIVALENT TARGETING FLUORESCENT TRACER

(71) Applicant: FLUOPTICS, GRENOBLE (FR)

(72) Inventor: Pascal GAYET, Grenoble (FR)

(21) Appl. No.: 15/576,257

(22) PCT Filed: May 24, 2016
(86) PCT No.: PCT/EP2016/061715

§ 371 Date: Nov. 21, 2017

(30) Foreign Application Priority Data
May 25, 2015 (FR) 1554669

(51) Int. Cl. A61K 49/00

(52) U.S. Cl. CPC A61K 49/0056; A61K 49/0032

(57) ABSTRACT

A fluorescent tracer comprises: a RAFT template, comprising the residues: Glycine-Proline-Lysine-Alanine-Lysine-Glycine-Proline-Lysine-Lysine-Lysine and having a mean plane defining an upper face having four lysine residues and a lower face having one lysine residue, four identical targeting molecules comprising the amino acids: Arginine-Glycine-Aspartic acid-Phenylalanine-Lysine (RGDKf), each of the molecules being fixed to a different lysine residue of the first upper face via an oxime bond, and a fluorophore S0456 fixed to the lower face of the mean plane via a spacer arm connecting the central carbon of the sequence of double bonds of the fluorophore and the lysine amino acid of the lower face of the decapeptide via an amide bond, the spacer arm being 5-(4-hydroxyphenyl)pentanoic acid.

The present invention relates to compounds such as probes with fluorescence in the near-infrared range. These compounds comprise targeting molecules for target tissues or organs, a fluorophore and a support on which the targeting molecules and the fluorophore are fixed.

### TECHNOLOGICAL BACKGROUND

Fluorescence imaging is growing rapidly in numerous surgical applications. Several medical devices are available on the market, which enable the detection and visualization of one of the only fluorophores which has marketing authorization, indocyanine green. However, this fluorophore is a molecule which is not specific for target tissues or organs, which restricts the field of use thereof.

Medical imaging is a promising technique in surgical procedures. It may, for example, serve to guide the surgeon during surgery. This technique relies on the administration of a fluorescent tracer to the patient. In the case in point, this is a fluorescent tracer which targets the tissues or organs requiring the surgical procedure.

The principle is based on illumination, by a light source, of a fluorescent tracer, administered to the patient beforehand, which is specific for target tissues or organs. The illumination has the effect of exciting said fluorescent tracer, which in turns emits radiation at a given wavelength. The main applications are in the near-infrared range, between 700 nm and 1000 nm. This is because this optical window corresponds to the range of wavelengths in which biological tissues absorb the least.

The advantage of fluorescent tracers is that they make it possible to combine properties of at least two classes of molecules, such as properties of fluorescence on the one hand and properties of targeting specific tissues or organs on the other.

With this in mind, a tracer, represented in FIG. 1, which fluoresces in the near-infrared range and is specific for the αᵥβ₃ integrin has been proposed.

The fluorescent tracer Tf1 comprises a molecular template 1, targeting molecules 3 and a fluorophore 4.

The molecular template 1 is a RAFT cyclic decapeptide, RAFT being an acronym standing for “regioselectively addressable functionalized template”.

It is a cyclic decapeptide comprising the sequence of amino acid residues: -Glycineₐ-Proline_{b}-Lysine_{c}-Alanine_{d}-Lysineₑ-Glycine_{f}-Proline_{g}-Lysineₕ-Lysineᵢ-Lysineⱼ-[-Gₐ-P_{b}-K_{c}-A_{d}-Kₑ-G_{f}-P_{g}-Kₕ-Kᵢ-Kⱼ], the sequences of the glycine and proline amino acid residues G_{a;f}; P_{b;g} forming bends 2 such that the configuration of the molecular template 1 has a mean plane Pm defining what is referred to as an upper face Fₛ comprising four lysine residues K_{c}, Kₑ, Kₕ and Kⱼ of the cyclic decapeptide, and what is referred to as a lower face Fᵢ, opposite to the upper face Fₛ, comprising the lysine residue Kᵢ.

Mean plane Pm is intended to mean the plane for which the sum of the distances between the mean plane Pm and the amino acid residues is minimal.

The RAFT cyclic decapeptide produced in this way enables presentation of multimeric targeting molecules, and may be associated in a controlled manner with two independent functional domains: one domain intended for targeting zones of interest, such as zones expressing integrins, and one domain for detection.

The targeting molecules 3 are cyclic pentapeptides comprising the sequence of amino acid residues -RGD-.

Numerous integrins interact with their protein substrates via the sequence of amino acid residues -RGD-, the acronym for “arginine-glycine-aspartic acid”. This RGD sequence is a common motif present on the majority of the proteins of the extracellular matrix. The cyclic pentapeptide molecules 3 are coupled to the four lysine amino acid residues K_{c}, Kₑ, Kₕ and Kⱼ of what is referred to as the upper face Fₛ of the molecular template 1 via oxime bonds.

The fluorophore 4 of the cyanine family fluoresces in the range of wavelengths of between 700 and 900 nm. It should be noted that, here, the fluorophore 4 is IRDye800 (registered trademark), but may also be cyanine 5 (registered trademark), Alexa fluor 700 (registered trademark) or a fluorophore developed specifically at 700 nm, denoted BM 105 (registered trademark).

The fluorophore 4 is linked to the lysine residue Kᵢ of what is referred to as the lower face Fᵢ of the molecular template 1 by forming an amide bond via an aliphatic group of one of the aromatic indole groups 4b of the fluorophore 4.

The fluorescent tracer Tf1 produced according to the known art has an absorption maximum at 781 nm and an emission maximum at 801 nm; it is particularly well-suited to the Fluobeam (registered trademark) imaging device from Fluoptics.

The process for producing the fluorescent tracer Tf1 according to the prior art comprises:

a first step of synthesizing the RAFT template 1 comprising:
a sub-step of synthesizing a linear decapeptide [-K(boc)-K(Alloc)-K(Boc)-P-G-K(Boc)-A-K(Boc)-P-G] on resin,
a sub-step of cyclizing the decapeptide in solution,
a sub-step of deprotecting the K(Boc)s,
a sub-step of grafting a protected oxyamine precursor,
a sub-step of deprotecting the K(Alloc)s,

a second step of synthesizing the targeting molecules 3; in the present case, the cyclic pentapeptides having the sequence of amino acid residues: Aspartic acid, phenylalanine, lysine, arginine and glycine [-D(tBu)-F-K(Alloc)-R(Pbf)-G-] 3,

a third step of coupling between the RAFT template 1 and targeting molecules 3, comprising:
a sub-step of cyclizing the pentapeptides in solution,
a sub-step of deprotecting K(Alloc),
a sub-step of grafting protected S,
a sub-step of deprotecting D, S and R, and
a sub-step of oxidizing S,

a fourth step of activating the fluorophore, and

a fifth step of coupling, subsequent to the third and fourth steps, between the RAFT template comprising the targeting molecule 3 and the fluorophore 4.

In practice, the fluorophore 4 is sold in the activated form, which makes it particularly unstable and reactive, especially with regard to amines.

The yields of this process are relatively low; especially the yield from coupling between the RAFT template 1 comprising the targeting molecules 3 and the fluorophore 4. Indeed, to obtain 15 g of fluorescent tracer Tf1 such as that described in FIG. 1, 410 g of targeting molecule 3, 75 g of RAFT template 1 and 7 g of fluorophore 4 are necessary.

The overall costs of producing a fluorescent tracer Tf1 according to the known art are very high, due both to the cost of purchasing the fluorophore 4 and also to the synthesis process which generates high losses of starting materials.

One aim of the present invention is to propose an alternative to the fluorescent tracer proposed according to the prior art, which overcomes the abovementioned problem.

Thus, the fluorescent tracer according to the invention is developed specifically to enable excellent compatibility with a near-infrared imaging device such as the Fluobeam (registered trademark) device by Fluoptics, and has a lower cost than that of the tracers proposed according to the prior art. Indeed, the Fluobeam (registered trademark) device comprises a laser for excitation at 750 nm, making it possible to obtain the fluorescence intensity maximum of the fluorescent tracer according to the invention.

### SUMMARY OF THE INVENTION

According to one aspect of the invention, a fluorescent tracer is proposed, comprising:

a cyclic decapeptide consisting of the sequence of the ten amino acid residues: -Glycine-Proline-Lysine-Alanine-Lysine-Glycine-Proline-Lysine-Lysine-Lysine, configured so as to define a mean plane defining a first upper face having four lysine amino acid residues and a second lower face having one lysine amino acid residue,

four identical targeting molecules, the targeting molecules being cyclic pentapeptides consisting of the sequence of amino acid residues: Arginine-Glycine-Aspartic acid-Phenylalanine-Lysine, each of the targeting molecules being fixed to a different lysine amino acid residue of the first upper face via an oxime bond, and

a fluorophore, 3,3-Dimethyl-2-[2-[2-chloro-3-[2-[1,3-dihydro-3,3-dimethyl-5-sulfo-1-(4-sulfobutyl)-2H-indol-2-ylidene]ethylidene]-1-cyclohexen-1-yl]ethenyl]-5-sulfo-1-(4-sulfobutyl)-3H-indolium hydroxide, inner salt, trisodium salt, having a sequence of double bonds with a central carbon, the fluorophore being fixed to the second lower face of the mean plane via a spacer arm connecting the central carbon of the sequence of double bonds of the fluorophore via an ether bond, and the lysine amino acid residue of the lower face of the decapeptide via an amide bond, the spacer arm being 5-(4-hydroxyphenyl)pentanoic acid.

The tracer thus proposed is well-suited to the Fluobeam (registered trademark) imaging device from Fluoptics, and has a quantum yield six times greater than that obtained with indocyanine green, currently in clinical use.

According to another aspect of the invention, a process for synthesizing a fluorescent tracer is proposed, the fluorescent tracer comprising:

a cyclic decapeptide consisting of the sequence of the ten amino acid residues: -Glycine-Proline-Lysine-Alanine-Lysine-Glycine-Proline-Lysine-Lysine-Lysine, configured so as to define a mean plane defining a first upper face having four lysine amino acid residues and a second lower face having one lysine amino acid residue,

four identical targeting molecules, the targeting molecules being cyclic pentapeptides consisting of the sequence of amino acid residues: Arginine-Glycine-Aspartic acid-Phenylalanine-Lysine, each of the targeting molecules being fixed to a different lysine amino acid residue of the first upper face via an oxime bond, and
a fluorophore, 3,3-Dimethyl-2-[2-[2-chloro-3-[2-[1,3-dihydro-3,3-dimethyl-5-sulfo-1-(4-sulfobutyl)-2H-indol-2-ylidene]ethylidene]-1-cyclohexen-1-yl]ethenyl]-5-sulfo-1-(4-sulfobutyl)-3H-indolium hydroxide, inner salt, trisodium salt, having a sequence of double bonds with a central carbon, the fluorophore S0456 being fixed to the second lower face of the mean plane via a spacer arm connecting the central carbon of the sequence of double bonds of the fluorophore via an ether bond, and the lysine amino acid residue of the decapeptide via an amide bond, the spacer arm being 5-(4-hydroxyphenyl)pentanoic acid,

the process comprising a step of fixing the fluorophore to the decapeptide via the spacer arm, prior to a step of fixing the targeting molecules, thereby making it possible to do away with the step of activating the fluorophore.

### LIST OF FIGURES

The invention will be better understood and other advantages will become apparent on reading the nonlimiting description which follows, and by virtue of the appended figures among which:

FIG. 1, already described, represents a fluorescent tracer according to the known art,

FIG. 2 represents the fluorescent tracer developed for the Fluobeam (registered trademark) imaging device from Fluoptics, according to the invention,

FIG. 3 is a superposition of the absorption spectra of the fluorescent tracers according to the known art and according to the invention,

FIG. 4 is a superposition of the emission spectra of the fluorescent tracers according to the known art and according to the invention,

FIGS. 5a and 5b demonstrate the tissue distribution of the fluorescent tracers according to the known art and according to the invention,

FIG. 6 represents the coupling reactions between the spacer arm and the fluorophore according to the invention,

FIGS. 7a and 7b represent reactions for synthesizing the RAFT template, and for coupling between the fluorophore provided with the spacer arm and the RAFT template, according to the invention,

FIG. 8 represents reactions for synthesizing the targeting molecules according to the invention,

FIG. 9 represents the coupling reaction between the fluorescent RAFT template and the targeting molecules via an amide bond, according to the invention.

### DETAILED DESCRIPTION

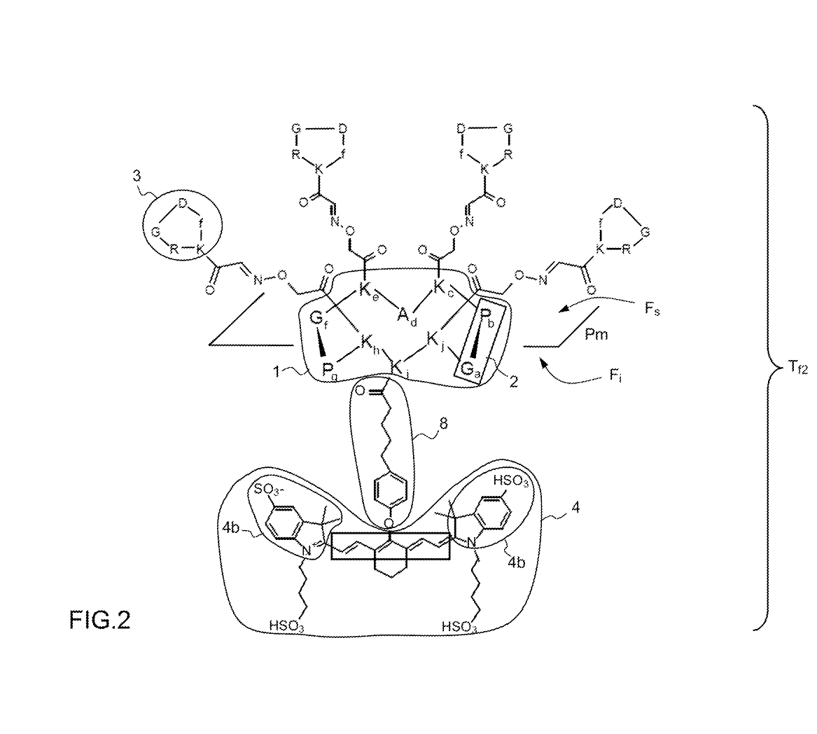

FIG. 2 represents the fluorescent tracer Tf according to the invention.

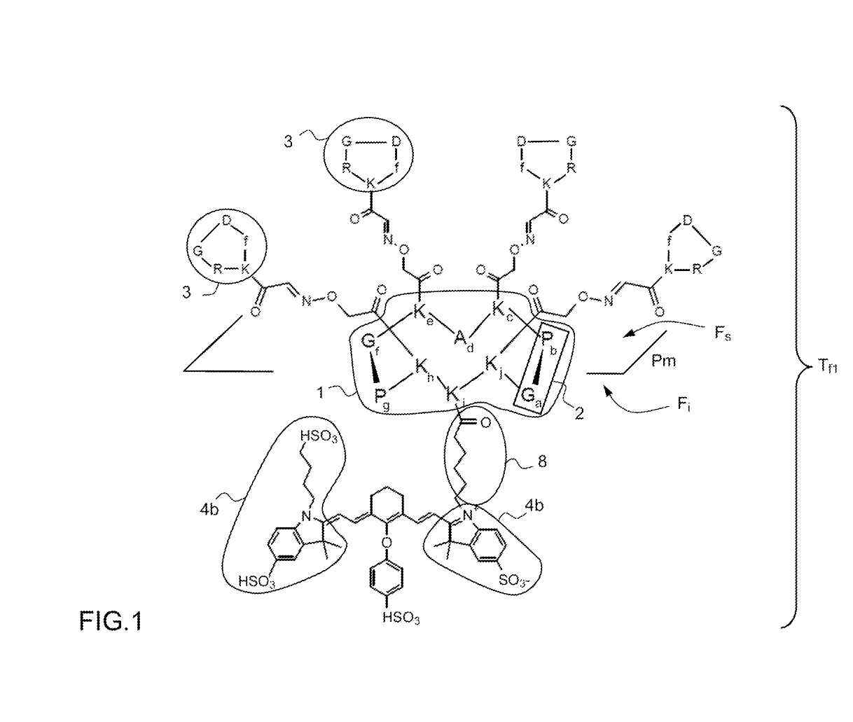

Like the tracer Tf1 of the prior art described in FIG. 1, the tracer Tf2 according to the invention comprises a molecular support 1 to which targeting molecules 3 and the fluorophore 4 are fixed.

The molecular template 1 or molecular support is a RAFT cyclic decapeptide comprising the sequence of ten amino acid residues: Glycine, Proline, Lysine, Alanine, Lysine, Glycine, Proline, Lysine, Lysine, Lysine -Gₐ-P_{b}-K_{c}-A_{d}-Kₑ-G_{f}-P_{g}-Kₕ-Kᵢ-Kⱼ. The sequences of amino acid residues: Glycine-Proline -G_{a;f}; P_{b;g}- constitute bends 2 defining a mean plane Pm. The molecular template 1 has a first upper face Fₛ having four lysine amino acid residues K_{c}, Kₑ, Kₕ and Kⱼ and a second lower face Fᵢ having a single lysine amino acid residue Kᵢ. The choice of a decapeptide as molecular template 1 enables the fixing of four targeting molecules 3.

The targeting molecules 3 are cyclic pentapeptides comprising the sequence RGD which is specific for integrin, and more specifically the sequence: Arginine-Glycine-Aspartic acid-Phenylalanine-Lysine-RGDfK-. Integrin, and more particularly the αᵥβ₃ integrin, which is the target of the abovementioned sequence, is overexpressed by neoangiogenic zones and by numerous human tumor cell lines.

The targeting molecules 3 are coupled to the four lysine amino acid residues K_{c}, Kₑ, Kₕ and Kⱼ of what is referred to as the upper face Fₛ of the molecular template 1 via oxime bonds.

The fluorophore 4 is S0456 (registered trademark) or 3,3-Dimethyl-2-[2-[2-chloro-3-[2-[1,3-dihydro-3,3-dimethyl-5-sulfo-1-(4-sulfobutyl)-2H-indol-2-ylidene]ethylidene]-1-cyclohexen-1-yl]ethenyl]-5-sulfo-1-(4-sulfobutyl)-3H-indolium hydroxide, inner salt, trisodium salt. The fluorophore S0456 4 belongs to the cyanine family and fluoresces in the range of wavelengths of between 700 and 900 nm.

The fluorophore S0456 is connected to the template via a spacer arm 8, 5-(4-hydroxyphenyl)pentanoic acid. This fluorophore has a carbon-based chain 4a enabling delocalization of the electrons. In addition, this fluorophore 4 S0456 has sulfonate groups on the aromatic groups 4b, conferring good solubility in aqueous phase on the fluorescent tracer Tf2.

The spacer arm 8 is fixed to the lower face Fᵢ of the molecular template 1 at a lysine amino acid residue K via an amide bond. The spacer arm 8 is fixed to the fluorophore 4 at the carbon in the place of the element chlorine via an ether bond and more specifically via an oxyphenyl bond which enables additional delocalization of the electrons over the phenyl group. Moreover, the ether bond barely modifies the wavelength of the emission maximum of the fluorophore 4, the wavelength of the emission maximum being reduced by 10 to 15 nm relative to the fluorophore 4 not fixed to the template 1 via the spacer arm 8.

The tracer according to the invention differs from the tracer according to the known art in that the fluorophore is connected to the template via a spacer arm 8 connecting a central carbon of the carbon-based chain to the lysine Kᵢ of the lower face Fᵢ of the template, which makes it possible to use less expensive fluorophores than those used according to the prior art.

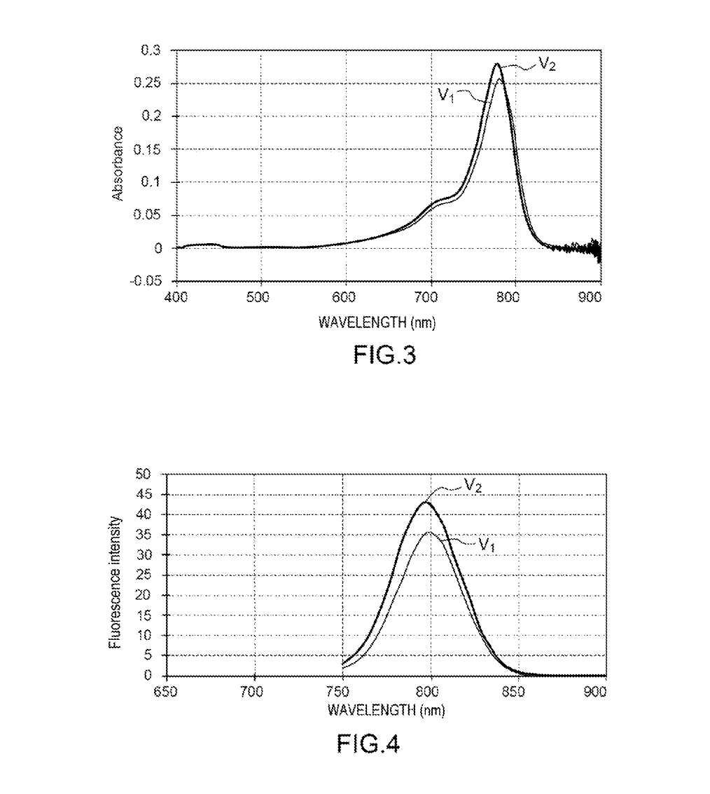

FIG. 3 is a superposition of the absorption spectrum of a fluorescent tracer according to the known art (curve V1) and of the fluorescent tracer according to the invention (curve V2). The two curves have an absorption peak, the maximum absorption values of which appear at a wavelength of 781 nm for a tracer Tf1 according to the known art and of 778 nm for the tracer Tf2 according to the invention.

Moreover, FIG. 4 is a superposition of the emission spectra of a fluorescent tracer Tf1 according to the known art (curve V1) and of the fluorescent tracer Tf2 according to the invention (curve V2). The two curves have a peak, the maximum emission values of which appear at a wavelength of 801 nm for a tracer Tf1 according to the known art and of 797 nm for the tracer Tf2 according to the invention.

FIGS. 3 and 4 do indeed show that the optical properties of the fluorescent tracer according to the known art Tf1 and according to the invention Tf2 are substantially equivalent. Thus, the tracer Tf2 according to the invention appears perfectly suited to the Fluobeam (registered trademark) imaging device from Fluoptics.

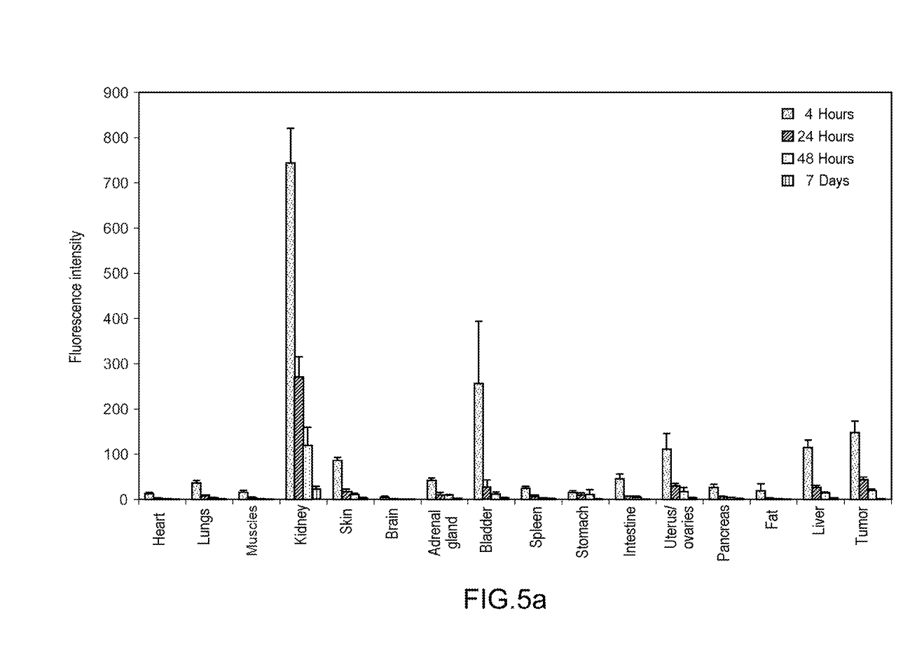

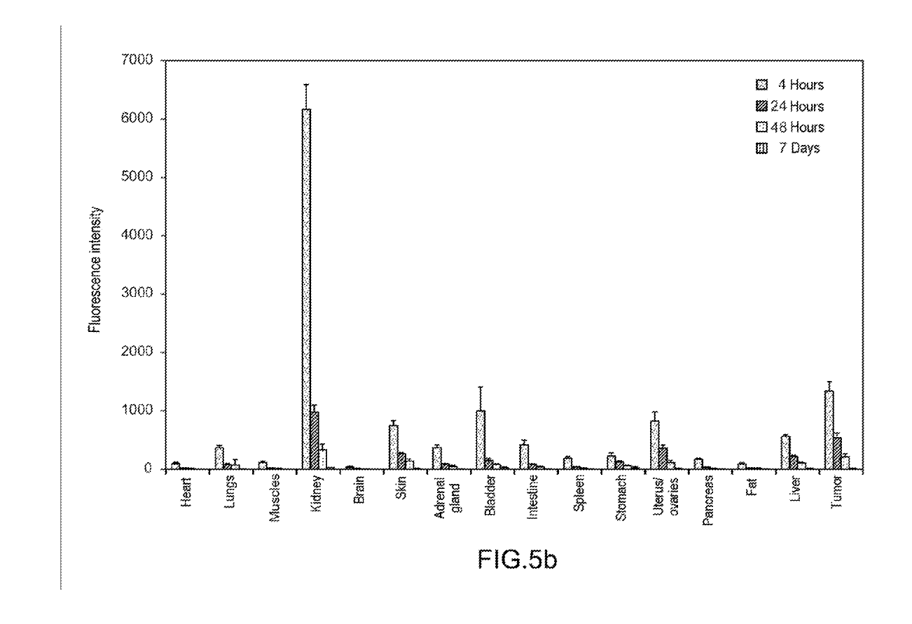

FIG. 5a illustrates the tissue distribution of a tracer Tf1 according to the known art, at different times post-injection.

In the case in point, the fluorescence was measured at times post-injection of 4 h, 24 h, 48 h then seven days and the organs or tissues studied were: the heart, the lungs, the muscles, the kidney, the skin, the brain, the adrenal glands, the bladder, the spleen, the stomach, the intestines, the ovaries and the uterus, the pancreas, fat, the liver and a subcutaneous murine mammary tumor (Ts/Apc).

The diagram shows that the fluorescence intensity is greatest at a time, post-injection of the tracer Tf1, of 4 h, and decreases after 24 h. After seven days, the fluorescence intensity in the organs and tissues is virtually zero.

In addition, the diagram shows that the tracer Tf1 according to the known art is particularly well-suited for targeting a tumor which is overexpressing the αᵥβ₃ integrin. Moreover, this diagram also shows a significant accumulation of the tracer Tf1 in the kidneys from 4 hours post-injection, demonstrating rapid renal elimination of the product.

FIG. 5b represents the fluorescence intensity of a tracer Tf2 according to the invention, in the same organs and tissues as those studied in FIG. 5a.

The tissue distribution of the tracer Tf2 according to the invention is similar to the distribution observed for the tracer Tf1 according to the known art.

Moreover, the affinity of the tracer Tf2 according to the invention is similar to the affinity of a tracer Tf1 such as Cy5-RAFT-(c[RGDfK])₄ according to the known art. This is explained by the fact that the targeting molecules are identical and represented in identical amounts in the tracer Tf1 according to the known art and the tracer Tf2 according to the invention.

On the other hand, the tracer Tf2 according to the invention has a much greater affinity than a monomeric tracer having the targeting molecule, represented in a single example.

According to another aspect of the invention, a process for producing the tracer Tf2, comprising a step of coupling the fluorophore 4 with the RAFT template 1 so as to form a fluorescent RAFT template prior to the step of coupling the targeting molecules 3, is proposed.

This production process reduces starting material losses, and more particularly losses of the RGD targeting molecules, and makes it possible to avoid the step of activating the fluorophore before the coupling step.

The process comprises:

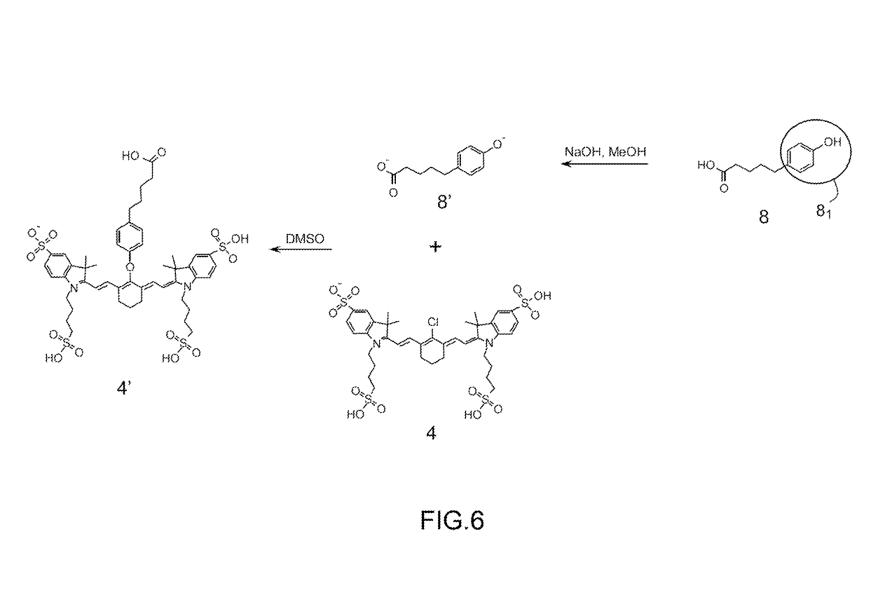

a first step, illustrated in FIG. 6, for preparing a modified fluorophore 4′ in which the spacer arm 8 is coupled to the fluorophore 4,

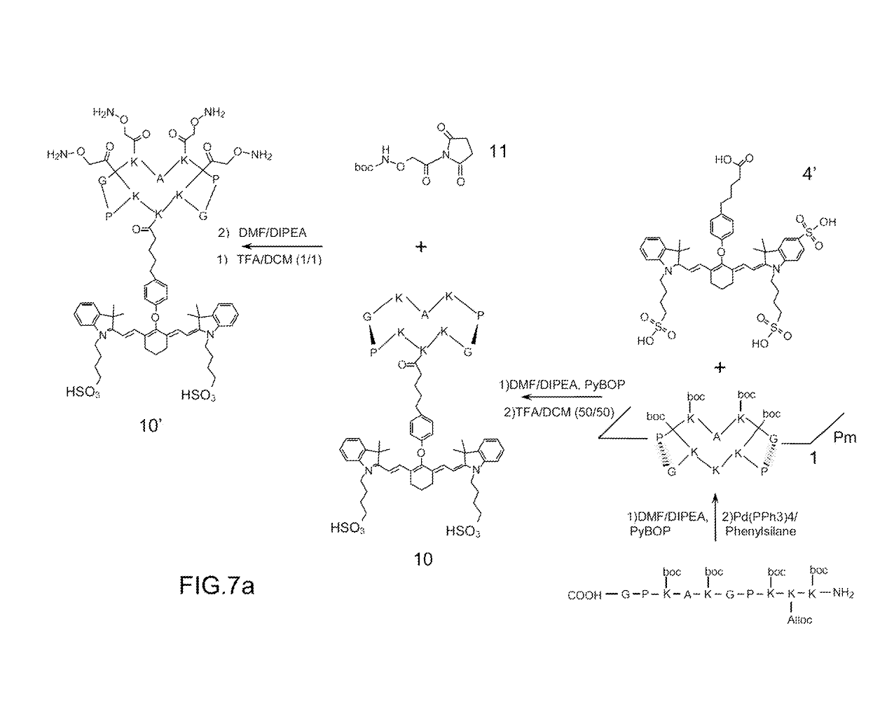

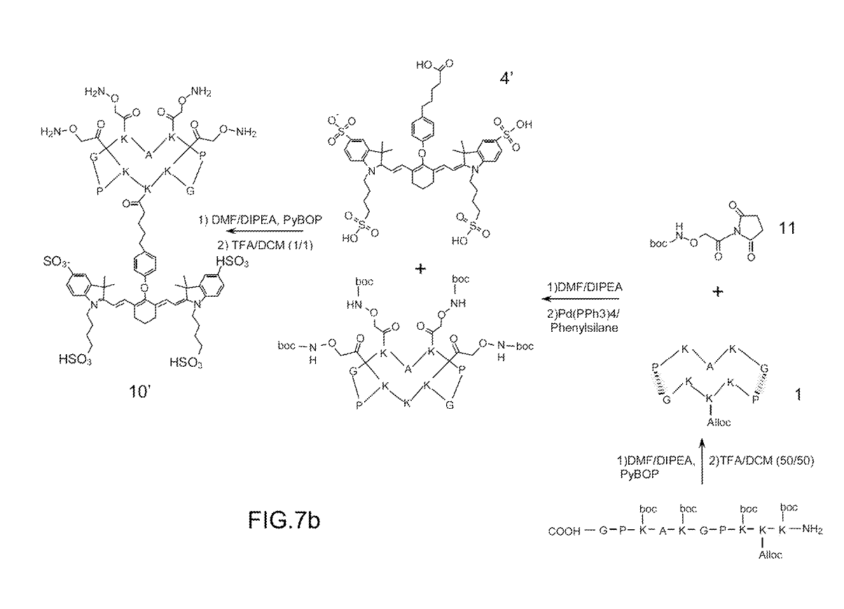

a second step, represented in FIG. 7a, in which the RAFT template 1 is synthesized,

a third step, represented in FIG. 7a, in which the RAFT template 1 is coupled to the modified fluorophore 4′ so as to form a fluorescent template 10,

a fourth step, illustrated in FIG. 7a, in which an oxime bond precursor 11 is grafted onto the RAFT template 1 on the lysine residues of what is referred to as the upper face Fₛ so as to form a modified fluorescent template 10′,

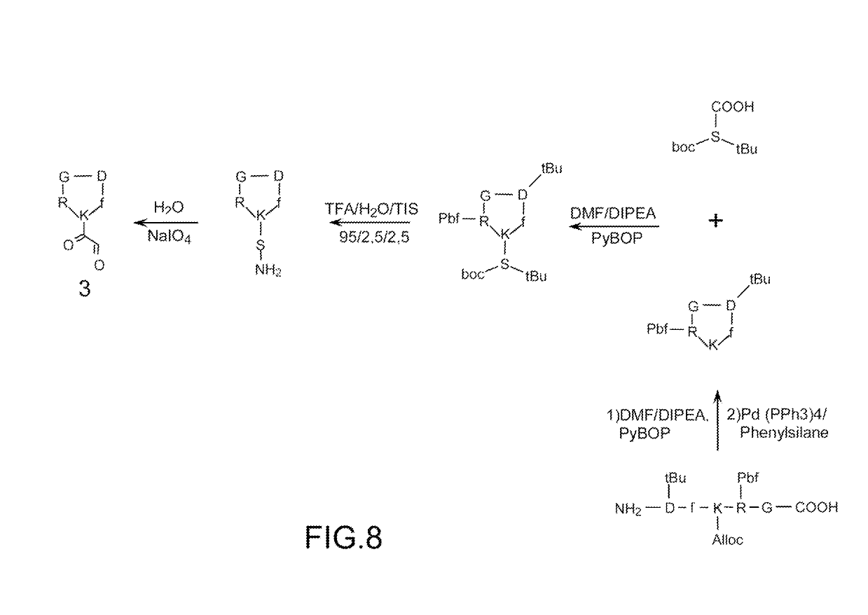

a fifth step, represented in FIG. 8, in which the targeting molecules 3 are synthesized, and

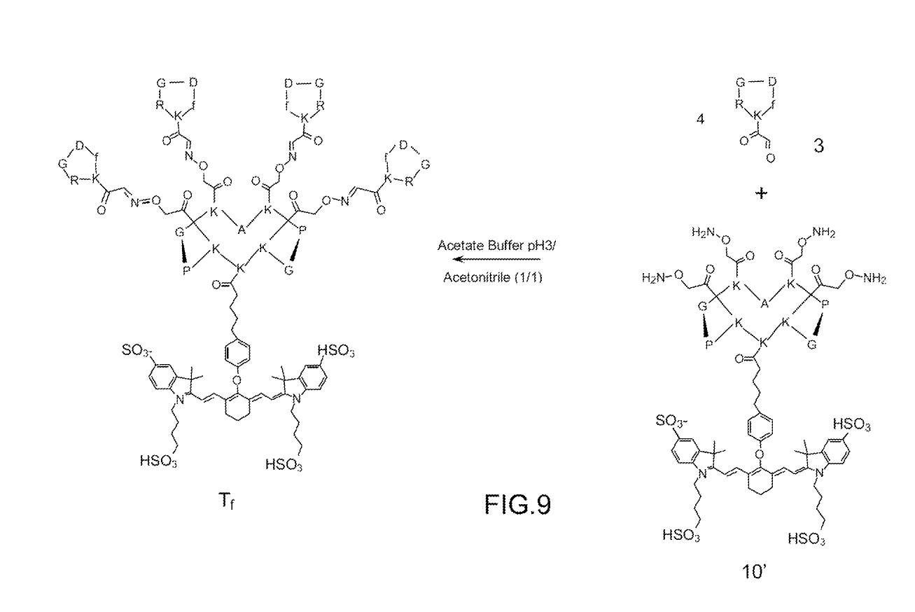

a sixth step, illustrated in FIG. 9, for coupling between the targeting molecules 3 and the modified fluorescent template 10′.

More specifically, FIG. 6 presents the steps for preparing the modified fluorophore 4′, described especially in the document by Hyun, H. et al, “c-GMP-compatible preparative scale of near-infrared fluorophores. Contrast Media Mol. Imaging, 2012, 7: 516”. The spacer arm 8, 5-(4-hydroxyphenyl)pentanoic acid, comprises a first phenol end group 8₈₁. The phenol is converted to phenolate, which is more reactive than phenol, in a solution of sodium hydroxide in methanol, to form a modified spacer arm 8′. The spacer arm 8′ obtained is then mixed with the fluorophore 4, in this case S0456 (or 3,3-Dimethyl-2-[2-[2-chloro-3-[2-[1,3-dihydro-3,3-dimethyl-5-sulfo-1-(4-sulfobutyl)-2H-indol-2-ylidene]ethylidene]-1-cyclohexen-1-yl]ethenyl]-5-sulfo-1-(4-sulfobutyl)-3H-indolium hydroxide, inner salt, trisodium salt) in DMSO (acronym for DiMethyl SulfOxide) to obtain the modified fluorophore 4′ consisting of the fluorophore 4 onto which the spacer arm 8′ is grafted.

FIG. 7a presents the second, third and fourth sub-steps of the process. Firstly, a linear decapeptide comprising the sequence of amino acid residues [-K(Boc)-K(Alloc)-K(Boc)-P-G-K(Boc)-A-K(Boc)-P-G-] is synthesized on resin, the Boc and Alloc groups being protecting groups so as to subsequently enable regioselective functionalization of the fluorescent template. After detachment from the resin, the decapeptide is cyclized and the lysine residue protected by the (Alloc) group is deprotected in the presence of palladium (Pd⁰) and phenylsilane so as to form a RAFT template 1 having a mean plane Pm. It should be noted that the third step and the fourth step may be switched around, as illustrated in FIG. 7b. In the case in point, firstly the Boc protecting groups on the lysine residues of what is referred to as the upper face Fₛ are cleaved in acid medium then protected oxyamine precursors 11 are grafted onto the lysine residues via an amide bond. The lysine residue located on the lower face Fᵢ protected by the (Alloc) group is deprotected so as to enable the grafting of the modified fluorophore 4′ to form a modified fluorescent template 10′.

The modified fluorophore 4′ is then grafted onto the deprotected lysine residue to form a fluorescent template 10. The Boc protecting groups on the lysine residues of what is referred to as the upper face Fₛ are cleaved in acid medium then a protected oxyamine precursor 11 is grafted onto the lysine residues via an oxime bond, to form the modified fluorescent RAFT template 10′.

It is interesting to note that the sub-step of activating the fluorophore 4, which is essential in the process according to the prior art, is not necessary in the process according to the invention.

FIG. 8 presents the steps for synthesizing the targeting molecule 3. Firstly, the linear pentapeptide comprising the sequence of amino acid residues RGD specific for integrins is synthesized on a resin. In the case in point, the pentapeptide comprises the sequence [-D(tBu)-f-K(Alloc)-R(Pbf)-G-]. After detaching the peptide from the resin, said peptide is cyclized then the lysine residue protected by the Alloc group is subsequently deprotected in the presence of palladium (Pd⁰) and phenylsilane. A protected serine residue is then grafted onto the lysine residue before total deprotection of the pentapeptide in acid medium. The alcohol function of the serine is then oxidized with sodium periodate in water, to obtain an aldehyde group.

FIG. 9 illustrates the step of coupling between the modified fluorescent template 10′ and the cyclic pentapeptides 3.

In the claimed process, the commercial fluorophore 4 is involved very early on in the process for synthesizing the fluorescent tracer Tf2 compared to the process of the prior art.

The requirements for purity and quality relating to starting materials incorporated into formulations intended for human administration involved early on in the synthesis process are less stringent than for starting materials involved at the end of the process, as is the case in the process of the prior art. In the case in point, the fluorophore 4 may therefore be of lesser quality and purity than those required during the synthesis according to the process of the prior art, which contributes significantly to lowering the purchase cost of the fluorophore 4.

Moreover, unlike the process described in the prior art, the step of activating the fluorophore prior to the step of coupling between the modified fluorophore 10′ and the RAFT template 1 is not necessary, which makes it possible to further reduce the costs of producing the fluorescent tracer Tf2 according to the invention.

Thus, the synthesis of 15 g of fluorescent tracer Tf2 requires 30 g of RAFT, 105 g of RGD and 13 g of fluorophore.

The fluorescent tracer Tf2 proposed in the present invention is suited for use with a fluorescence imaging device of Fluobeam (registered trademark) type; it makes it possible to significantly reduce costs, on the one hand by enabling the use of less expensive fluorophore but also by improving the production process, thereby making it possible to do away with reaction steps which had hitherto been essential in the process proposed according to the prior art.

1. A fluorescent tracer, comprising:
a cyclic decapeptide consisting of the sequence of the ten amino acid residues: -Glycine-Proline-Lysine-Alanine-Lysine-Glycine-Proline-Lysine-Lysine-Lysine (-Gₐ-P_{b}-K_{c}-A_{d}-Kₑ-G_{f}-P_{g}-Kₕ-Kᵢ-Kⱼ-) configured so as to define a mean plane defining a first upper face having four lysine amino acid residues and a second lower face having one lysine amino acid residue,
four identical targeting molecules, the targeting molecules being cyclic pentapeptides consisting of the sequence of amino acid residues: Arginine-Glycine-Aspartic acid-Phenylalanine-Lysine (RGDKf), each of the targeting molecules being fixed to a different lysine amino acid residue of the first upper face via an oxime bond, and
a fluorophore, 3,3-Dimethyl-2-[2-[2-chloro-3-[2-[1,3-dihydro-3,3-dimethyl-5-sulfo-1-(4-sulfobutyl)-2H-indol-2-ylidene]ethylidene]-1-cyclohexen-1-yl]ethenyl]-5-sulfo-1-(4-sulfobutyl)-3H-indolium hydroxide, inner salt, trisodium salt, having a sequence of double bonds with a central carbon, the fluorophore S0456 being fixed to the second lower face of the mean plane via a spacer arm connecting the central carbon of the sequence of double bonds of the fluorophore via an ether bond, and the lysine amino acid residue of the lower face of the decapeptide via an amide bond, the spacer arm being 5-(4-hydroxyphenyl)pentanoic acid.

2. A process for synthesizing a fluorescent tracer, the fluorescent tracer comprising:
a cyclic decapeptide consisting of the sequence of the ten amino acid residues: -Glycine-Proline-Lysine-Alanine-Lysine-Glycine-Proline-Lysine-Lysine-Lysine (-Gₐ-P_{b}-K_{c}-A_{d}-Kₑ-G_{f}-P_{g}-Kₕ-Kᵢ-Kⱼ-) configured so as to define a mean plane defining a first upper face having four lysine amino acid residues and a second lower face having one lysine amino acid residue,
four identical targeting molecules, the targeting molecules being cyclic pentapeptides consisting of the sequence of amino acid residues: Arginine-Glycine-Aspartic acid-Phenylalanine-Lysine (-RGDfK-), each of the targeting molecules being fixed to a different lysine amino acid residue of the first upper face via an oxime bond, and
a fluorophore, 3,3-Dimethyl-2-[2-[2-chloro-3-[2-[1,3-dihydro-3,3-dimethyl-5-sulfo-1-(4-sulfobutyl)-2H-indol-2-ylidene]ethylidene]-1-cyclohexen-1-yl]ethenyl]-5-sulfo-1-(4-sulfobutyl)-3H-indolium hydroxide, inner salt, trisodium salt, having a sequence of double bonds with a central carbon, the fluorophore being fixed to the second lower face of the mean plane via a spacer arm connecting the central carbon of the sequence of double bonds of the fluorophore via an ether bond, and the lysine amino acid residue of the decapeptide via an amide bond, the spacer arm being 5-(4-hydroxyphenyl)pentanoic acid,
the process comprising a step of fixing the fluorophore to the decapeptide via the spacer arm prior to a step of fixing the targeting molecules.
